(19) United States
(12) Patent Application Publication
(10) Pub. No.: US 2022/0375588 A1
(43) Pub. Date: Nov. 24, 2022

(54) PROCEDURAL SEDATION MONITORING SYSTEM AND METHOD

(71) Applicant: Edward Lehman, Las Vegas, NV (US)

(72) Inventor: Edward Lehman, Las Vegas, NV (US)

(21) Appl. No.: 17/683,485

(22) Filed: Mar. 1, 2022

Related U.S. Application Data
(60) Provisional application No. 63/156,158, filed on Mar. 3, 2021.

(51) Int. Cl. G16H 40/63; G05B 19/042

(52) U.S. Cl. CPC G16H 40/63; G05B 19/042; A61B 5/746

(57) ABSTRACT

A procedural sedation monitoring system increases awareness of medical providers of physiologic parameters of a patient throughout a medical procedure and includes a patient monitoring system having a plurality of patient sensors disposed in an operative engagement with the patient. A processor assembly includes a patient monitoring signal receiver and signal processor to receive and analyze a plurality of patient monitoring signals. An environmental control assembly includes an environmental control device and the processor assembly transmits an environmental control signal to the environmental control device to control operation thereof and to alter an aspect of the environment based on the signal. A sensory feedback assembly includes a sensory feedback device wearable by medical providers and having a sensory feedback generator providing a sensory feedback sensation to medical providers based on the sensory feedback signal to alert medical providers of physiologic parameters of the patient throughout the medical procedure.

### CROSS-REFERENCE TO RELATED APPLICATIONS

This application claims the benefit of U.S. Provisional Patent Application Ser. No. 63/156,158 filed on Mar. 3, 2021, which is incorporated by reference herein in its entirety.

### FIELD OF THE INVENTION

The present invention relates generally to a procedural sedation monitoring system, and a method for procedural sedation monitoring.

### BACKGROUND OF THE INVENTION

Even the most routine of medical procedures comes with an amount of risk. It is common for a local anesthetic to be utilized for many procedures, including minor surgery. For more serious and/or involved procedures, anesthesia is used to sedate the patient, which adds yet another risk factor to the overall procedure. Common forms of anesthesia include general anesthesia and procedural anesthesia, among others, and each comes with its own patient monitoring criteria in order to minimize the risk to the patient.

Procedural sedation is often touted as safe, but it is known to be a dangerous medical procedure. In fact, even in the hands of an experienced anesthesiologist, the American Society of Anesthesiologists (ASA) closed-claims database reveals that procedural sedation cases are potentially just a dangerous as general anesthesia cases. The highest risk category in these cases involves over-sedation and cardiorespiratory depression. The risk is most certainly higher in the hands of non-anesthesia sedation providers, as their level of expertise in recognizing the signs of over-sedation and/or cardiorespiratory depression is substantially lower.

Furthermore, it is also known that the majority of procedural sedation procedures are performed in an ambulatory surgery center (ASC), where regulatory compliance may be more lax than in a full hospital environment. Deep procedural sedation at an ASC has increased in incidence with consumer demand for patient comfort. In addition, the types and duration of procedures being performed with procedural sedation have increased as technology has improved capabilities. For example, ultrasound technology has improved and many conditions/diseases which formerly required surgery with general anesthesia can now be treated with less invasive tools, such that the procedure is performed while the patient is under procedural sedation. Some examples of such medical procedures performed with procedural sedation include endobronchial ultrasound (“EBUS”), transesophageal echocardiogram (“TEE”), gastrointestinal endoscopic ultrasound procedures, and interventional radiology procedures, just to name a few.

Currently, procedural sedation, such as may be employed during a routine colonoscopy, requires patient monitoring in accordance with the requirements defined by the ASA. More in particular, and as set forth by the ASA, a sedated patient must be constantly monitored during a medical procedure with at least the following: an electrocardiogram or ECG, a pulse oximeter, and a blood pressure monitor. Sensors capable of monitoring all three are typically wired to a monitoring device, which is usually a device having a screen showing visible signals and graphs and emitting an audible signal, typically, a beeping sound. These visual and audible indications are directed to a person standing or sitting close to the device.

It is understood, of course, that patient monitoring occurs and/or is required during other types of medical procedures as well including surgery without sedation; surgery performed with the patient under sedation ranging from light to moderate to deep sedation; total intravenous sedation; monitored anesthesia care, and/or general anesthesia, just to name a few.

However, as anyone with any experience in an operating theater or an ASC knows, medical personnel in a medical procedure room become so accustomed to the sights and sounds that they pay little to no attention to the display screen or the beeping sound, defined and recognized as alarm fatigue. Further, the aforementioned monitoring modalities, namely, an ECG, a pulse oximeter, and a blood pressure monitor which, once again, typically include a wired connection to the monitoring device, introduce a significant delay in indicating the actual real-time status of a patient's medical condition called physiologic signal latency and propagation delay, i.e., the length of time it takes for a signal to reach its destination. As just one example, it is believed that in some cases, a delay of as much as several minutes may exist between a patient's oxygen intake significantly decreasing, e.g., as may occur if the patient stops breathing, and the fact of this condition actually being reflected on the monitoring device screen and/or by the acoustic, i.e., beeping, feedback.

As a result of the foregoing, it is believed that many patient's consenting to procedural sedation for supposedly safe and often minimally invasive or even some non-invasive procedures are at considerable risk of permanent damage, and perhaps even death, as a result of partial respiratory or cardio failure which goes unnoticed by medical personnel during a supposedly safe and routine medical procedure, until it was too late.

Accordingly, there is an established need for a solution to one or more of the aforementioned problems.

### SUMMARY OF THE INVENTION

The present invention is generally directed to a procedural sedation monitoring system, and a method for procedural sedation monitoring.

In a first implementation of the invention, a procedural sedation monitoring system is provided to inform a medical provider of at least one physiologic parameter of a patient throughout a medical procedure performed in a controlled environment, such a system may comprise: a patient monitoring system having at least one patient sensor disposed in an operative engagement with the patient throughout the medical procedure; a processor assembly including a patient monitoring signal receiver, the at least one patient sensor transmitting at least one patient monitoring signal indicative of the at least one physiologic parameter to the patient monitoring signal receiver throughout the medical procedure; the processor assembly further comprising a patient signal processor to analyze the at least one patient monitoring signal and to generate at least one environmental control signal based on the at least one patient monitoring signal; an environmental control assembly comprising at least one environmental control device disposed in communication with the controlled environment; and the processor assembly transmitting the at least one environmental control signal to the at least one environmental control device to control operation thereof.

In a second aspect, the procedural sedation monitoring system can include at least one patient sensor comprising one of an electrocardiogram sensor, a pulse oximeter sensor, a blood pressure sensor, an impedance cardiography sensor, an acoustic respiration sensor, or a temperature sensor.

In another aspect, the procedural sedation monitoring system may have a patient monitoring system comprises a plurality of patient sensors.

In a further aspect, the procedural sedation monitoring system can include at least some of a plurality of sensors comprising a different one of an electrocardiogram sensor, a pulse oximeter sensor, a blood pressure sensor, an impedance cardiography sensor, an acoustic respiration sensor, or a temperature sensor.

In one other aspect, the procedural sedation monitoring system may have at least one of a plurality of sensors comprising an impedance cardiography sensor or an acoustic respiration sensor.

In yet another aspect, the procedural sedation monitoring system can include a. processor assembly further comprising an environmental control signal transmitter to transmit at least one environmental control signal to at least one environmental control device.

In still one further aspect, the procedural sedation monitoring system may have at least one environmental control device comprising a sound generator, a light generator, a thermal control unit, or a visual display generator.

In yet one other aspect, the procedural sedation monitoring system can include an environmental control assembly comprising a plurality of environmental control devices disposed in communication with a controlled environment.

In still another aspect, the procedural sedation monitoring system may have at least some of a plurality of environmental control devices comprising a different one of a sound generator, a light generator, a thermal control unit, or a visual display generator.

In yet one further aspect, a procedural sedation monitoring system is provided to inform of a medical provider of at least one physiologic parameter of a patient throughout a medical procedure and may comprise: a patient monitoring system having at least one patient sensor disposed in an operative engagement with the patient throughout the medical procedure; a processor assembly including a patient monitoring signal receiver, the at least one patient sensor transmitting at least one patient monitoring signal indicative of the at least one physiologic parameter to the patient monitoring signal receiver throughout the medical procedure; the processor assembly further comprising a patient signal processor to analyze at least one patient monitoring signal and to generate a sensory feedback signal based on the at least one patient monitoring signal; a sensory feedback assembly comprising a sensory feedback device disposed in communication with the medical provider; and the processor assembly transmitting the sensory feedback signal to the sensory feedback device to control operation thereof and to alert the medical provider of the at least one physiologic parameter of the patient throughout the medical procedure, wherein the processor assembly further comprises a sensory feedback signal transmitter to transmit the sensory feedback signal to the sensory feedback device.

In still one other aspect, the procedural sedation monitoring system may have a sensory feedback device comprising a sensory feedback harness dimensioned to be donned by a medical provider, the sensory feedback harness comprising a sensory feedback generator to provide a sensory feedback sensation to the medical provider based on a sensory feedback signal to alert the medical provider of the at least one physiologic parameter of the patient throughout the medical procedure.

In yet another aspect, the procedural sedation monitoring system can include a sensory feedback harness further comprising a sensory feedback signal receiver to receive a sensory feedback signal from a sensory feedback signal transmitter.

In still one further aspect, the procedural sedation monitoring system may have a sensory feedback device comprising a sensory feedback wristband dimensioned to be donned by the medical provider, the sensory feedback wristband comprising a sensory feedback generator to provide a sensory feedback sensation to the medical provider based on a sensory feedback signal to alert the medical provider of at least one physiologic parameter of the patient throughout the medical procedure.

In yet one other aspect, the procedural sedation monitoring system can include a sensory feedback wristband further comprising a sensory feedback signal receiver to receive a sensory feedback signal from a sensory feedback signal transmitter.

In yet another aspect, a procedural sedation monitoring system is provided to inform a medical provider of a plurality of physiologic parameter of a patient throughout a medical procedure performed in a controlled environment and may comprise: a patient monitoring system having a plurality of patient sensors disposed in an operative engagement with the patient throughout the medical procedure; a processor assembly including a patient monitoring signal receiver, the plurality of patient sensors transmitting a plurality of patient monitoring signals indicative of the plurality of physiologic parameters to the patient monitoring signal receiver throughout the medical procedure; the processor assembly further comprising a patient signal processor to analyze the plurality of patient monitoring signals and to generate at least one environmental control signal and at least one sensory feedback signal based on the plurality of patient monitoring signals; an environmental control assembly comprising at least one environmental control device disposed in communication with the controlled environment; the processor assembly transmitting at least one environmental control signal to at least one environmental control device to control operation thereof; a sensory feedback assembly comprising a sensory feedback device disposed in communication with the medical provider comprising a sensory feedback generator to provide a sensory feedback sensation to the medical provider based on the sensory feedback signal to alert the medical provider of at least one physiologic parameter of the patient throughout the medical procedure; and the processor assembly transmitting the sensory feedback signal to the sensory feedback device to control operation thereof.

These and other objects, features, and advantages of the present invention will become more readily apparent from the attached drawings and the detailed description of the embodiments, which follow.

### BRIEF DESCRIPTION OF THE DRAWINGS

The embodiments of the invention will hereinafter be described in conjunction with the appended drawings provided to illustrate and not to limit the invention, where like designations denote like elements, and in which:

FIG. 1 presents a diagrammatic representation of one illustrative embodiment of a procedural sedation monitoring system including a patient monitoring assembly and a processor assembly, in accordance with the present invention;

FIG. 2 presents a block diagram representative of one illustrative embodiment of a procedural sedation monitoring system, in accordance with the present invention;

FIG. 3 presents a diagrammatic representation of one illustrative embodiment of a procedural sedation monitoring system, in accordance with the present invention;

FIG. 4 presents a diagrammatic representation of one illustrative embodiment of a procedural sedation monitoring system including a processor assembly and a sensory feedback assembly, in accordance with the present invention;

FIG. 5 presents a diagrammatic representation of another illustrative embodiment of a procedural sedation monitoring system including a processor assembly and a sensory feedback assembly, in accordance with the present invention; and

FIG. 6 presents a block diagram representative of one illustrative embodiment of a method for procedural sedation monitoring, in accordance with the present invention.

Like reference numerals refer to like parts throughout the several views of the drawings.

### DETAILED DESCRIPTION

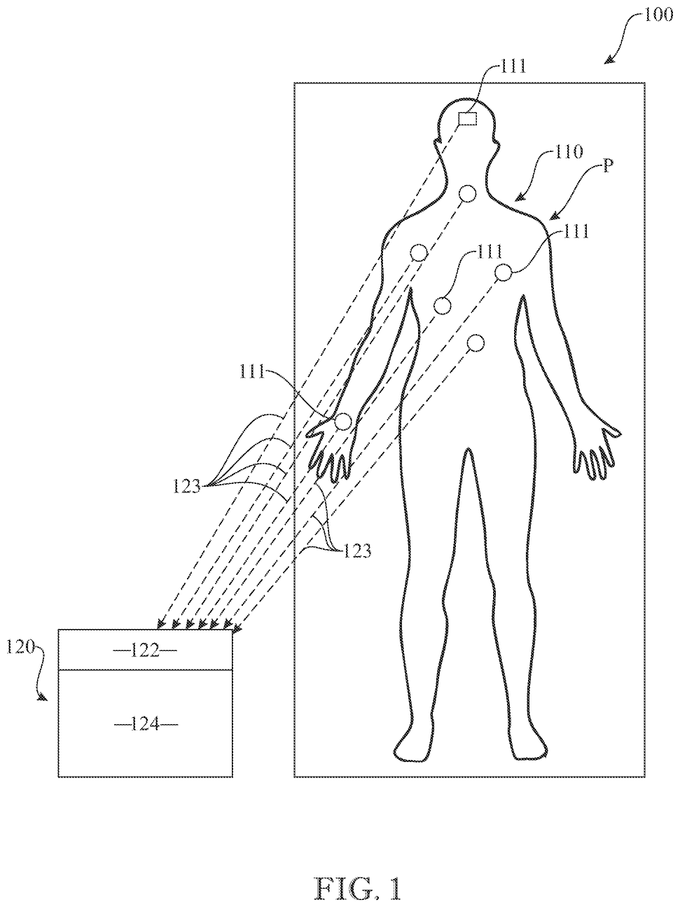

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “top”, “bottom” “left”, “right”, “front”, “rear”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present invention is generally directed to a procedural sedation monitoring system, and a method for procedural sedation monitoring. It is, of course, understood that present monitoring system and method may be utilized while performing other types of medical procedures as well including surgery without sedation; surgery performed with the patient under sedation ranging from light to moderate to deep sedation; total intravenous sedation; monitored anesthesia care, and/or general anesthesia, just to name a few.

Referring initially to FIG. 1, presented therein is a diagrammatic representation of one illustrative embodiment of a procedural sedation monitoring system 100 including a patient monitoring assembly 110 and a processor assembly 120, in accordance with the present invention. A patient monitoring assembly 110 in accordance with the present invention includes at least one patient sensor 111 disposed in an operative engagement with the patient P so as to measure at least one physiologic parameter of the patient P throughout a medical procedure. In at least one embodiment, the medical procedure is performed while the patent is under procedural sedation. As used herein, the duration of a “medical procedure” can include baseline monitoring for a period of time prior to the procedure, continuous monitoring during the medical procedure, as well as monitoring for a period of time while in recovery from the procedure.

As may be seen from FIG. 1, in at least one embodiment, a patient monitoring assembly 110 includes a plurality of patient sensors 111 disposed in an operative engagement with a patient's P, and more in particular, with various portions of a patient's body while the patient is positioned on an operating table, gurney, or similar patient support surface within a controlled environment in which a medical procedure is to be performed, such as, by way of example only, an operating theater or an outpatient clinic. The plurality of patient sensors 111 of a patient monitoring assembly 110 in accordance with at least one embodiment of the present invention include at least an electrocardiogram or ECG sensor 112, a pulse oximeter sensor 113, and a blood pressure sensor 114, such as are required by the American Society of Anesthesiologists, as noted above. A patient monitoring assembly 110 may also include one or more additional sensors to detect other physiologic parameters of a patient P including but not limited to, an electroencephalogram or EEG sensor 117 or a temperature sensor 118, just to name a few.

In at least one embodiment, included among a plurality of patient sensors 111 of a patient monitoring assembly 110 in accordance with the present invention are an impedance cardiography sensor 115. An impedance cardiography sensor 115 in accordance with the present invention can detect minor changes in fluids and volumes in the thoracic cavity as result of cardiac hemodynamics, which also produce changes in the thoracic impedance thereby allowing estimation of parameters related to the mechanical function of the heart such as cardiac output, stroke volume, systolic time ratio, and the time of ejection of the left ventricle or the pre-ejection period. More importantly, an impedance cardiography sensor 115 may provide an indication of an impending disruption to a patient's normal heart function in advance of such an indication from an ECG sensor 112 and/or a blood pressure sensor 114, thus allowing a medical provider MP to take such corrective measures as may be needed sooner than later.

In at least one further embodiment, the plurality of patient sensors 111 of a patient monitoring assembly 110 comprises an acoustic respiration sensor 116, operatively positioned so as to measure the sound of air passing through a patient's glottis throughout a medical procedure. As will be appreciated, and similar to the measurements provided by an impedance cardiography sensor 115, an acoustic respiration sensor 116 may provide an indication of impending respiratory distress in the patient before such an indication is measured and transmitted by a pulse oximeter sensor 113. In some cases, this advance indication may be minutes ahead of a measurable decrease in a patient's blood oxygen level, minutes which in many cases may well prove to be life-saving as they will allow a medical provider MP to react sooner, and take necessary corrective measures to prevent respiratory failure and save a patient's life.

As before, a procedural sedation monitoring system 100 in accordance with at least one embodiment of the present invention further comprises a processor assembly 120. With reference once again to FIG. 1, the processor assembly 120 includes a patient monitoring signal receiver 122. In at least one embodiment, a patient monitoring signal receiver 122 is disposed in or proximate to a controlled environment in which a medical procedure requiring procedural sedation is to be performed. Specifically, a patient monitoring signal receiver 122 is positioned so as to receive one or more patient monitoring signals 123 each corresponding to one or more patient sensors 111 operatively engaging a patient P during a medical procedure. More in particular, each patient sensor 111 transmits a patient monitoring signal 123 indicative of at least one physiologic parameter of the patient P being monitored throughout a medical procedure. In at least one embodiment, each of a plurality of patient monitoring signals 123 may be transmitted through traditional hardwired electrical lines disposed between each of a plurality of patient sensors 111 and a patient monitoring signal receiver 122 of the processor assembly 120. In accordance with at least one further embodiment, each of a plurality of patient monitoring signals 123 are transmitted remotely from corresponding ones of a plurality of patient sensors 111 to a patient monitoring signal receiver 122, thereby eliminating potential entanglement and/or disengagement of one or more of the plurality of patient sensors 111 by medical providers while performing a medical procedure on a patient P.

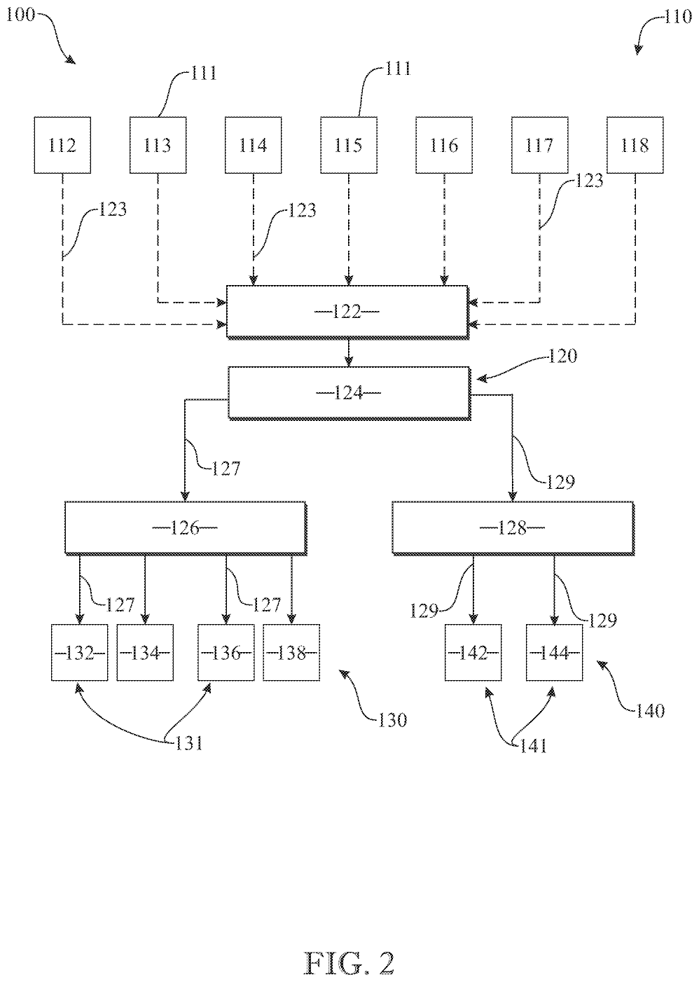

Looking next to FIG. 2, presented therein is a block diagram representative of one illustrative embodiment of a procedural sedation monitoring system 100, in accordance with the present invention. As before, a procedural sedation monitoring system 100 includes a patient monitoring assembly 110 comprising at least one patient sensor 111. As shown in the illustrative embodiment of FIG. 2, in at least one embodiment, a patient monitoring assembly 110 comprises a plurality of patient sensors 111. In accordance with at least one embodiment of the present invention, a plurality of patient sensors 111 include one or more of an electrocardiogram or ECG sensor 112, a pulse oximeter sensor 113, a blood pressure sensor 114, an impedance cardiography sensor 115, an acoustic respiration sensor 116, an electroencephalogram or EEG sensor 117, and a temperature sensor 118, as are represented diagrammatically in the illustrated embodiment of FIG. 2. As will be appreciated by those of skill in the art, a patient monitoring assembly 110 in accordance with the present procedural sedation monitoring system 100 may comprise additional patient sensors 111 as may be warranted by a particular condition specific to a patient P undergoing a medical procedure and or, the specific monitoring requirements of a particular medical procedure itself. As will be further appreciated by those of skill in the art, it is well within the scope and intent of the present invention for a patient monitoring assembly 110 to only include some of the patient sensors 111 identified above and in FIG. 2 by reference numerals 112 through 118.

A procedural sedation monitoring system 100 in accordance with at, least one embodiment of the present invention further comprises a processor assembly 120. As before, a processor assembly 120 comprises a patient monitoring signal receiver 122 configured and disposed to receive at least one patient monitoring signal 123 transmitted from at least one patient sensor 111 disposed in an operative engagement with a patient P undergoing a medical procedure. In at least one further embodiment, and as is shown best in FIGS. 1 and 2, a patient monitoring signal receiver 122 in accordance with the present invention is disposed and configured to receive a plurality of patient monitoring signals 123 transmitted from each of a plurality of patient sensors 111 disposed in an operative engagement with a patient P undergoing a medical procedure while under procedural sedation. A processor assembly 120 in accordance with one embodiment of the present invention includes a patient signal processor 124. With reference again to FIG. 2, a patient signal processor 124 of a processor assembly 120 is disposed in a communicative relation with a patient monitoring signal receiver 122. More in particular, the patient signal processor 124 is disposed in communication with a patient monitoring signal receiver 122 which transfers a plurality of patient monitoring signals 123, received from a plurality of patient sensors 111 disposed in an operative engagement with a patient P, to the patient signal processor 124 throughout a medical procedure.

A patient signal processor 124 in at least one embodiment continuously receives and analyzes a plurality of patient monitoring signals 123 received from one or more patient sensors 111 disposed in an operative engagement with the patient P throughout a medical procedure, each indicative of at least one physiologic parameter of the patient P. More in particular, a patient signal processor 124 analyzes the plurality of patient monitoring signals 123 to determine if the data representative of one or more physiologic parameters of a patient P are within acceptable operative ranges for a patient under procedural sedation during a medical procedure.

In one embodiment, a processor assembly 120 further comprises an environmental control signal transmitter 126. In such an embodiment, a patient signal processor 124 analyzes a plurality of patient monitoring signals 123 and generates a corresponding plurality of environmental control signals 127 which are relayed to an environmental control signal transmitter 126 throughout a medical procedure in which a patient P is undergoing procedural sedation. Provided a patient monitoring signal 123 is indicative of a physiologic parameter within an acceptable operative range for a patient P under procedural sedation during a medical procedure, the patient signal processor 124 is programmed to generate a corresponding environmental control signal 127 indicative of a normal physiologic parameter of the patient P. However, in the event that a patient monitoring signal 123 is indicative of a physiologic parameter outside of an acceptable operative range for a patient P under procedural sedation during a medical procedure, the patient signal processor 124 is programmed to generate a corresponding environmental control signal 127 indicative of an abnormal physiologic parameter of the patient P. Furthermore, in the event the patient monitoring signal 123 is indicative of a physiologic parameter in a critical range, the patient signal processor 124 is programmed to generate a corresponding environmental control signal 127 indicative of an alarm condition for a physiologic parameter of the patient P.

In another embodiment, a processor assembly 120 further comprises a sensory feedback signal transmitter 128. In such an embodiment, a patient signal processor 124 analyzes a plurality of patient monitoring signals 123 and generates a corresponding plurality of sensory feedback signals 129 which are relayed to a sensory feedback signal transmitter 128 throughout a medical procedure in which a patient P is undergoing procedural sedation. Provided a patient monitoring signal 123 is indicative of a physiologic parameter within an acceptable operative range for a patient under procedural sedation during a medical procedure, the patient signal processor 124 is programmed to generate a corresponding sensory feedback signal 129 indicative of a normal physiologic parameter of the patient P. However, and similar to an embodiment comprising an environmental control signal transmitter 126, in the event that a patient monitoring signal 123 is indicative of a physiologic parameter outside of an acceptable operative range for a patient P under procedural sedation during a medical procedure, the patient signal processor 124 is programmed to generate a corresponding sensory feedback signal 129 indicative of an abnormal physiologic parameter of the patient P. Also similar to an embodiment comprising an environmental control signal transmitter 126, in the event the patient monitoring signal 123 is indicative of a physiologic parameter in a critical range, the patient signal processor 124 is programmed to generate a corresponding sensory feedback signal 129 indicative of an alarm condition for a physiologic parameter of the patient P.

A procedural sedation monitoring system 100 in accordance with at least one embodiment of the present invention comprises an environmental control assembly 130 disposed in communication with a processor assembly 120. More in particular, an environmental control assembly 130 comprises at least one environmental control device 131 disposed in a communicative arrangement with a controlled environment in which a medical procedure is performed on a patient P undergoing procedural sedation. Further, and as described above, a plurality of environmental control signals 127 are generated by a patient signal processor 124 and relayed to an environmental control signal transmitter 126, in the manner disclosed above, and transmitted by the environmental control signal transmitter 126 to at least one environmental control device 131, to control the operation of the at least one environmental control device 131 throughout the medical procedure.

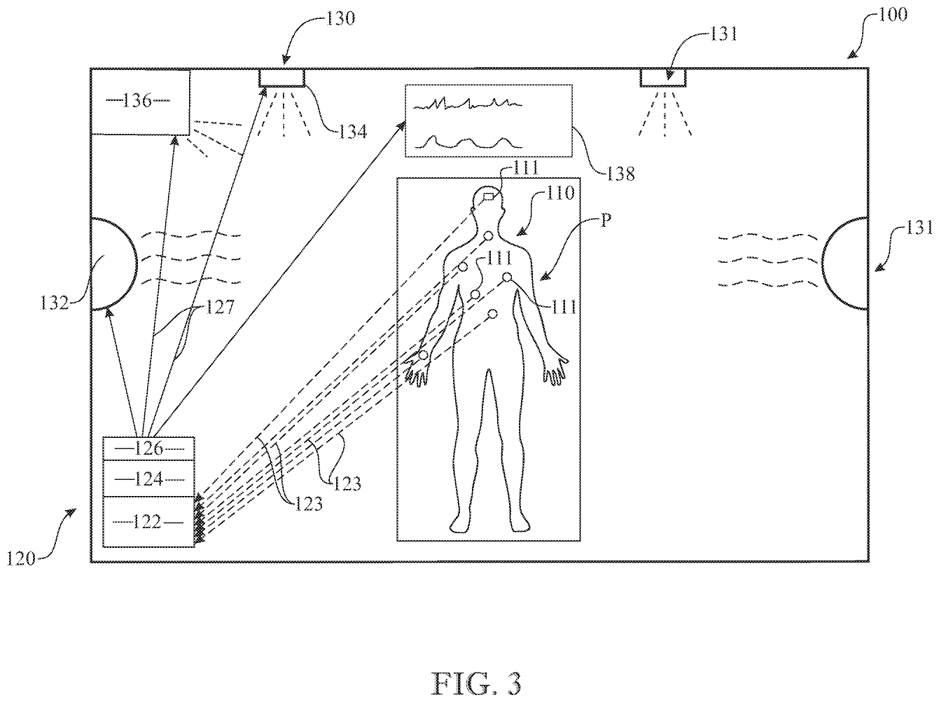

As shown best in FIGS. 2 and 3, an environmental control signal transmitter 126 is disposed in communication with an environmental control assembly 130 comprising a plurality of environmental control devices 131. In one embodiment, an environmental control device 131 comprises a sound generator 132 configured and disposed to generate and transmit an audible sound within a controlled environment during a medical procedure indicative of at least one physiologic parameter of a patient P undergoing the medical procedure. More in particular, when a measured physiologic parameter is within an acceptable operative range for a patient P under procedural sedation during a medical procedure, the patient signal processor 124 generates an environmental control signal 127 which is transmitted to the sound generator 132 thereby causing the sound generator 132 to produce an audible but ambient sound in the controlled environment which is audible to medical providers MP therein indicative of a physiologic parameter within an acceptable range. In the event a measured physiologic parameter is outside an acceptable operative range for a patient P under procedural sedation during a medical procedure, the patient signal processor 124 generates an environmental control signal 127 which causes the sound generator 132 to produce a different audible sound in the controlled environment audible to medical providers MP therein which is indicative of a physiologic parameter outside of an acceptable range. As just one example, a different audible sound may be an increase or decrease in volume or an increase or decrease in pitch or tone such as to indicate to the medical providers MP that a physiologic parameter either exceeds or is below an acceptable operative range, respectively. Likewise, a different sound may comprise a high or low level audible alarm sound audible to medical providers MP in a controlled environment indicating a high or low alarm condition has been measured for at least one physiologic parameter of the patient P being measured by a patient monitoring assembly 110 during a medical procedure while the patient P is undergoing procedural sedation.

An environmental control assembly 130 in accordance with at least one further embodiment of the present procedural sedation monitoring system 100 may comprise additional environmental control devices 131. In one embodiment, an environmental control device 131 comprises a light generator 134. Similar in operation to sound generator 132, a light generator 134 may generate and transmit an ambient lighting condition indicative of a measured physiologic parameter within an acceptable operative range for a patient P, as well as increasing or decreasing an ambient lighting condition in the event a measured physiologic parameter of the patient P falls outside of an acceptable operative range. Also similar to a sound generator 132, in the event one or more measured physiologic parameter is indicative of an alarm condition, a light generator 134 may generate and transmit a visible alarm signal such as sudden increase in intensity and/or flashing, so as to alert medical providers MP within the controlled environment of the alarm condition of the patient P. In similar fashion, an environmental control assembly may comprise a thermal control unit 136 operative to raise or lower a temperature in a controlled environment indicative of a measured physiologic parameter of the patient P undergoing a medical procedure therein.

In at least one embodiment, an environmental control assembly 130 further comprises a visual display generator 138. More in particular, in one embodiment a visual display generator 138 resembles the familiar, almost ubiquitous, patient monitor present in nearly every operating theater as well as in intensive care units wherein the physiologic parameters of the patient P are closely monitored by medical providers MP. As such, a visual display generator 138 includes a graphically and audible display of a plurality of measured physiologic parameters of a patient P such as respiration rate, temperature, blood pressure, pulse rate, oxygen levels, etc. In accordance with the present procedural sedation monitoring system 100, a visual display generator 138 graphically displays a plurality of measured physiologic parameters of a patient P on a plurality of visual displays strategically disposed throughout a controlled environment such that at least one of the plurality of visual displays is visible to each medical provider MP present in a controlled environment regardless of their proximity or orientation relative to the patient P.

While shown throughout the figures located in the same controlled environment in which a medical procedure is performed on a patient P undergoing procedural sedation, it is understood to be within the scope and intent of the present invention for a plurality of environmental control signals 127 generated by a patient signal processor 124 and relayed to an environmental control signal transmitter 126, in the manner disclosed above, to be transmitted by the environmental control signal transmitter 126 to at least one environmental control device 131 of an environmental control assembly 130 which is disposed and operable in a location remote of the controlled environment in which a medical procedure is performed on a patient P. As such, a medical provider in a location remote of the controlled environment in which a medical procedure is performed on a patient P can monitor the patient and/or the progress of the procedure. Alternatively, a medical provider may actually perform the medical procedure from a remote location, such as, by way of example, via robotic surgery. It is further understood that a plurality of environmental control signals 127 may be transmitted by the environmental control signal transmitter 126 to each of a plurality of environmental control devices 131 of each of a plurality of environmental control assemblies 130 each disposed and operable in a different remote location from the controlled environment in which a medical procedure is performed on a patient P.

A procedural sedation monitoring system 100 in accordance with at least one other embodiment of the present invention comprises a sensory feedback assembly 140 disposed in communication with a processor assembly 120. More in particular, a sensory feedback assembly 140 comprises at least one sensory feedback device 141 disposed in a communicative arrangement with a medical provider MP while a medical procedure is performed on a patient P undergoing procedural sedation. In one further embodiment, a plurality of sensory feedback signals 129 are generated by a patient signal processor 124 and relayed to a sensory feedback signal transmitter 128, in the manner disclosed above, and transmitted by the sensory feedback signal transmitter 128 to at least one sensory feedback device 141, to control the operation of the at least one sensory feedback device 141 throughout the medical procedure.

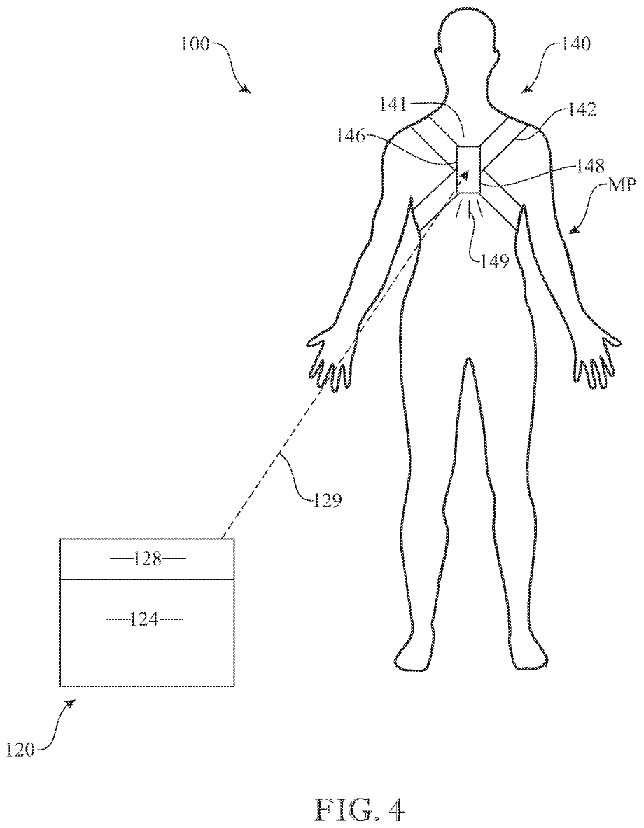

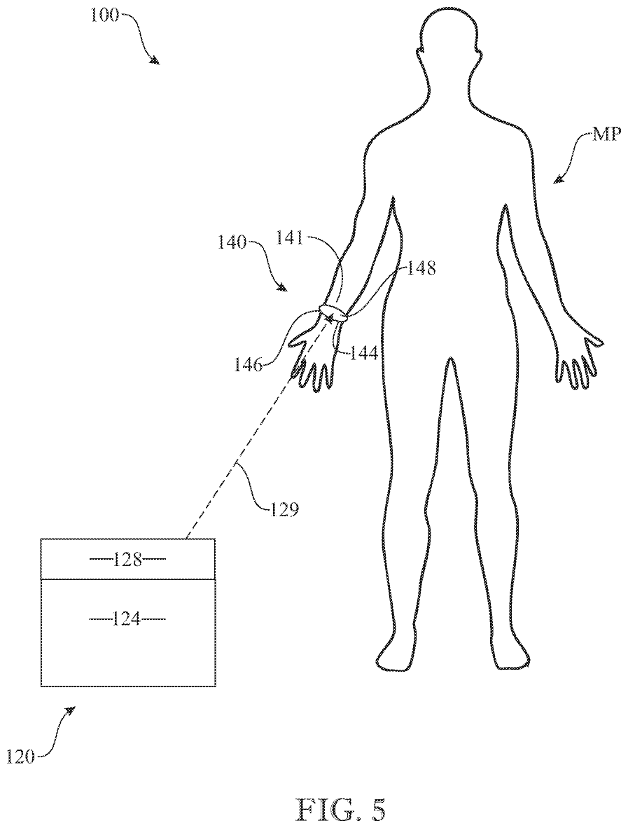

As shown best in FIGS. 2, 4 and 5, a sensory feedback signal transmitter 128 is disposed in communication with a sensory feedback assembly 140 comprising a plurality of sensory feedback devices 141. In one embodiment, a sensory feedback device 141 comprises a sensory feedback harness 142 configured and dimensioned to be worn around a portion of medical provider's MP body during a medical procedure, such as, across the medical provider's chest, as is shown by way of example in the illustrative embodiment of FIG. 4. In one alternative embodiment, a sensory feedback device 141 comprises a sensory feedback wristband 144 configured and dimensioned to be worn around a wrist of at least one medical provider MP during a medical procedure, such as is shown by way of example in the illustrative embodiment of FIG. 5.

A sensory feedback device 141 in one embodiment includes a sensory feedback signal receiver 146 configured to receive a plurality of sensory feedback signals 129 which are relayed to a sensory feedback signal transmitter 128 by a patient signal processor 124, in the manner described above. Furthermore, a sensory feedback signal transmitter 128 transmits a plurality of sensory feedback signals 129 to a sensory feedback signal receiver 146 of a sensory feedback device 141, throughout a medical procedure in which a patient P is undergoing procedural sedation. A medical provider donning a sensory feedback device 141 may be located in the same controlled environment in which the patient P is undergoing a medical procedure, or the medical provider, or medical providers, may be located at one or more remote locations from where the medical procedure is being performed on the patient P. Once again, this permits one or more medical providers to monitor the patent and/or the progress of the procedure, as well as permitting one or more medical providers the ability to actually perform the medical procedure remotely, once again, such as via robotic surgery.

In at least one further embodiment, a sensory feedback device 141 comprises a sensory feedback generator 148 which is configured to generate a sensory feedback sensation 149 indicative of at least one physiologic parameter of a patient P undergoing a medical procedure. More in particular, when a measured physiologic parameter is within an acceptable operative range for a patient P under procedural sedation during a medical procedure, the patient signal processor 124 generates a sensory feedback signal 129 which is transmitted to a sensory feedback signal receiver 146 of a sensory feedback device 141, thereby causing a sensory feedback generator 148 to produce a sensory feedback sensation 149 which is sensed by the medical provider MP wearing the sensory feedback device 141 and is indicative of a physiologic parameter within an acceptable range. As one example, a sensory feedback sensation 149 may comprise a steady audible ambient sound, a steady ambient pulsation or vibration, and/or a steady warm or cool sensation, just to name a few, each indicative to the medical provider MP that one or more physiologic parameters of a patient P are within an acceptable range.

In the event a measured physiologic parameter is outside an acceptable operative range for a patient P under procedural sedation during a medical procedure, the patient signal processor 124 generates sensory feedback signal 129 which causes the sensory feedback generator 148 to produce a sensory feedback sensation 149 which is indicative of a physiologic parameter outside of an acceptable range. As just one example, an audible ambient sound may increase or decrease in volume, pitch and/or tone so as to indicate to the medical provider MP that a physiologic parameter either exceeds or is below an acceptable operative range, respectively. Alternatively, a steady ambient pulsation or vibration may increase or decrease in intensity and/or a steady warming or cooling sensation may become hotter or colder, once again, so as to indicate to the medical provider MP that a physiologic parameter has either exceeded or fallen below an acceptable operative range, respectively. Likewise, a different sound, pulsation or vibration or temperature sensation may be utilized to indicate to a medical provider MP that a high or low alarm condition has been measured for at least one physiologic parameter of the patient P by a patient monitoring assembly 110 during a medical procedure while the patient P is undergoing procedural sedation. As before, by alerting medical providers MP at the very onset of a deviation into a potential alarm condition, they are able to react quickly and take actions necessary to either avoid the alarm condition or to being actions needed to alleviate the alarm condition in the patient, either of which may prove to be life-saving measures.

With reference once again to FIG. 2, in at least one embodiment of a procedural sedation monitoring system 100 in accordance with the present invention, a processor assembly 120 comprises an environmental control signal transmitter 126 and a sensory feedback signal transmitter 128 each disposed in communication with a patient signal processor 124. As such, the patient signal processor 124 in the embodiment of FIG. 2 receives and analyzes a plurality of patient monitoring signals 123 received from a plurality of patient monitoring sensors 111, via a patient monitoring signal receiver 122, and generates a corresponding plurality of environmental control signals 127 which are relayed to an environmental control signal transmitter 126 and a corresponding plurality of sensory feedback signals 129 which are relayed to a sensory feedback signal transmitter 128 throughout a medical procedure in which a patient P is undergoing procedural sedation.

As will be appreciated from the foregoing, the combination of an environmental control assembly 130 and a sensory feedback assembly 140 in accordance with at least one embodiment of the present procedural sedation monitoring system 100 all but assures that one or more medical provider MP conducting a medical procedure on a patient P within a controlled environment will be informed of one or more measured physiologic parameters of the patient P throughout the medical procedure. More importantly, the medical providers MP will be informed of one or more measured physiologic parameters of the patient P with sufficient time to take corrective measures as may be needed in the event one or more physiologic parameters of the patient P fall outside of an acceptable operative range and/or are indicative of a life-threatening alarm condition in the patient P, thereby significantly decreasing the instances of irreparable harm and/or death of patients P during related to procedural sedation medical procedures.

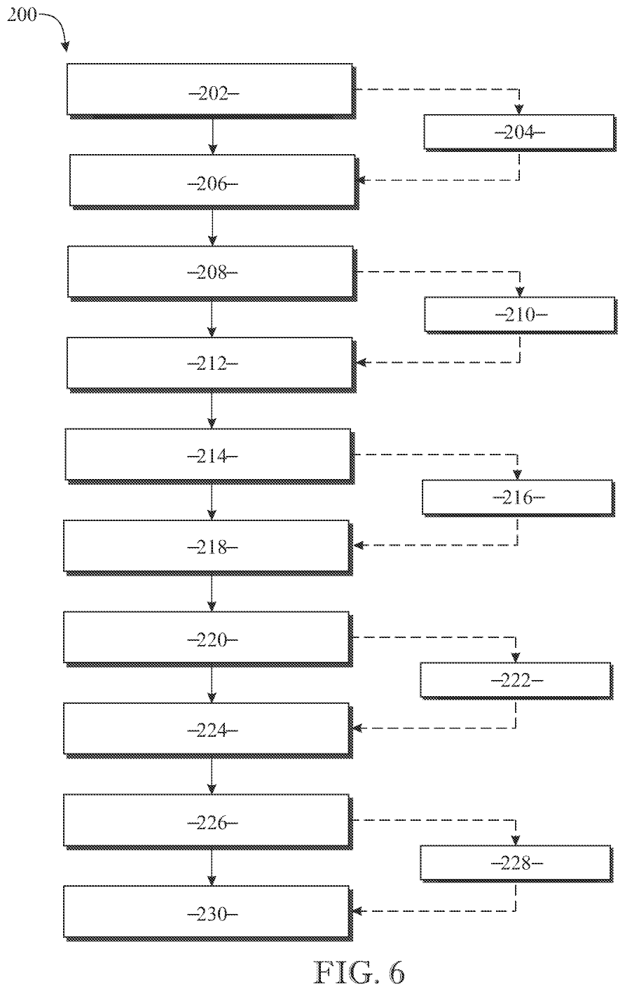

As indicated above, the present invention is further directed to a method of procedural sedation monitoring. FIG. 6 presents a block diagram of one illustrative embodiment of a method for procedural sedation monitoring, generally as shown as 200, in accordance with the present invention.

To begin, a method of procedural sedation monitoring 200 in accordance with the present invention begins with measuring at least one patient physiologic parameter 202 throughout a medical procedure. In at least one embodiment, the present method 200 includes measuring a plurality of patient physiologic parameters 202 throughout the medical procedure. A method for procedural sedation monitoring 200 further comprises analyzing a plurality of patient physiologic data 206 which is generated by measuring a plurality of patient physiologic parameters 202. In at least one embodiment, the present method 200 further comprises the step of transmitting a plurality of patient physiologic parameter data 204, such as is represented by dashed lines in FIG. 6.

After analyzing a plurality of patient physiologic data 206, the present method of procedural sedation monitoring 200 includes generating one or more environmental control signals 208 based at least in part on the plurality of patient physiologic data. The present method 200 further includes operating at least one environmental control device 212 via one or more environmental control signals. In at least one embodiment, the present method 200 includes operating a plurality of environmental control devices 212 via a plurality of environmental control signals. As shown in FIG. 6, a method for procedural sedation monitoring 200 in accordance with at least one embodiment of the present the invention includes transmitting one or more environmental control signals 210, also shown via dashed lines therein.

In at least one embodiment, the method of procedural sedation monitoring 200 further comprises generating an environmental control device alarm signal 214, wherein one or more of the plurality of patient physiologic parameters are outside of a normal or acceptable range for a patient undergoing procedural sedation. More in particular, if one or more of the plurality of patient physiologic parameters are indicative of impending respiratory or cardiac failure, or are otherwise in a critical range of another measured physiologic parameter during the procedural sedation, an environmental control device alarm signal is generated, and the present method 200 further includes operating an environmental control device alarm 218 so as to alert medical providers of the patient's condition such that corrective actions may be implemented without delay. In at least one embodiment, the present method further includes transmitting one or more environmental control device alarm signals 216 to one or more environmental control device alarms to effect operation of the same.

At least one further embodiment of a method for procedural sedation monitoring 200 in accordance with the present invention comprises generating one or more sensory feedback signals 220 once again, based at least in part on the plurality of patient physiologic data. With reference once again to the illustrative embodiment of FIG. 6, the present method 200 further comprises operating a sensory feedback device 224 via one or more sensory feedback signal. In yet another embodiment, the present method for procedural sedation monitoring 200 also includes transmitting one or more sensory feedback signals 222 to one or more sensory feedback devices, thereby actuating operation thereof.

Still one further embodiment of the present method 200 includes generating a sensory feedback device alarm signal 226. As in the case with generating an environmental control device alarm signal 214, if one or more of the plurality of patient's physiologic parameters are indicative of impending respiratory or cardiac failure or other measured critical physiologic parameter of the patient during procedural sedation, a sensory feedback alarm signal is generated, and the present method 200 further includes operating a sensory feedback device alarm 230 so as to alert medical providers of the patient's condition such that corrective actions may be implemented without delay. In at least one embodiment, the present method further includes transmitting one or more sensory feedback device alarm signals 228 to one or more sensory feedback device alarms to effect operation of the same.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

What is claimed is:

1. A procedural sedation monitoring system to inform a medical provider of at least one physiologic parameter of a patient throughout a medical procedure performed in a controlled environment, said system comprising:
a patient monitoring system having at least one patient sensor disposed in an operative engagement with the patient throughout the medical procedure;
a processor assembly including a patient monitoring signal receiver, said at least one patient sensor transmitting at least one patient monitoring signal indicative of the at least one physiologic parameter to said patient monitoring signal receiver throughout the medical procedure;
said processor assembly further comprising a patient signal processor to analyze said at least one patient monitoring signal and to generate at least one environmental control signal based on said at least one patient monitoring signal;
an environmental control assembly comprising at least one environmental control device disposed in communication with the controlled environment; and
said processor assembly transmitting said at least one environmental control signal to said at least one environmental control device to control operation thereof.

2. The system as recited in claim 1, wherein said at least one patient sensor comprises one of an electrocardiogram sensor, a pulse oximeter sensor, a blood pressure sensor, an impedance cardiography sensor, an acoustic respiration sensor, or a temperature sensor.

3. The system as recited in claim 1, wherein said patient monitoring system comprises a plurality of patient sensors.

4. The system as recited in claim 3, wherein at least some of said plurality of sensors comprise a different one of an electrocardiogram sensor, a pulse oximeter sensor, a blood pressure sensor, an impedance cardiography sensor, an acoustic respiration sensor, or a temperature sensor.

5. The system as recited in claim 3, wherein at least one of said plurality of sensors comprises an impedance cardiography sensor or an acoustic respiration sensor.

6. The system as recited in claim 1, wherein said processor assembly further comprises an environmental control signal transmitter to transmit said at least one environmental control signal to said at least one environmental control device.

7. The system as recited in claim 6, wherein said at least one environmental control device comprises a sound generator, a light generator, a thermal control unit, or a visual display generator.

8. The system as recited in claim 1, wherein said environmental control assembly comprises a plurality of environmental control devices disposed in communication with the controlled environment.

9. The system as recited in claim 8, wherein at least some of said plurality of environmental control devices comprise a different one of a sound generator, a light generator, a thermal control unit, or a visual display generator.

10. A procedural sedation monitoring system to inform a medical provider of at least one physiologic parameter of a patient throughout a medical procedure, said system comprising:
a patient monitoring system having at least one patient sensor disposed in an operative engagement with the patient throughout the medical procedure;
a processor assembly including a patient monitoring signal receiver, said at least one patient sensor transmitting at least one patient monitoring signal indicative of the at least one physiologic parameter to said patient monitoring signal receiver throughout the medical procedure;
said processor assembly further comprising a patient signal processor to analyze said at least one patient monitoring signal and to generate a sensory feedback signal based on said at least one patient monitoring signal;
an environmental control assembly comprising at least one environmental control device disposed in communication with the controlled environment;
a sensory feedback assembly comprising a sensory feedback device disposed in communication with the medical provider; and
said processor assembly transmitting said sensory feedback signal to said sensory feedback device to control operation thereof and to alert the medical provider of the at least one physiologic parameter of the patient throughout the medical procedure.

11. The system as recited in claim 10, wherein said at least one patient sensor comprises one of an electrocardiogram sensor, a pulse oximeter sensor, a blood pressure sensor, an impedance cardiography sensor, an acoustic respiration sensor, or a temperature sensor.

12. The system as recited in claim 10, wherein said patient monitoring system comprises a plurality of patient sensors.

13. The system as recited in claim 12, wherein at least some of said plurality of sensors comprise a different one of an electrocardiogram sensor, a pulse oximeter sensor, a blood pressure sensor, an impedance cardiography sensor, an acoustic respiration sensor, or a temperature sensor.

14. The system as recited in claim 12, wherein at least one of said plurality of sensors comprises an impedance cardiography sensor or an acoustic respiration sensor.

15. The system as recited in claim 10, wherein said processor assembly further comprises a sensory feedback signal transmitter to transmit said sensory feedback signal to said sensory feedback device.

16. The system as recited in claim 15, wherein said sensory feedback device comprises a sensory feedback harness dimensioned to be donned by the medical provider, said sensory feedback harness comprising a sensory feedback generator to provide a sensory feedback sensation to the medical provider based on said sensory feedback signal to alert the medical provider of the at least one physiologic parameter of the patient throughout the medical procedure.

17. The system as recited in claim 16, wherein said sensory feedback harness further comprises a sensory feedback signal receiver to receive said sensory feedback signal from said sensory feedback signal transmitter.

18. The system as recited in claim 15, wherein said sensory feedback device comprises a sensory feedback wristband dimensioned to be donned by the medical provider, said sensory feedback wristband comprising a sensory feedback generator to provide a sensory feedback sensation to the medical provider based on said sensory feedback signal to alert the medical provider of the at least one physiologic parameter of the patient throughout the medical procedure.

19. The system as recited in claim 18, wherein said sensory feedback wristband further comprises a sensory feedback signal receiver to receive said sensory feedback signal from said sensory feedback signal transmitter.

20. A procedural sedation monitoring system to inform a medical provider of a plurality of physiologic parameter of a patient throughout a medical procedure performed in a controlled environment, said system comprising:
a patient monitoring system having a plurality of patient sensors disposed in an operative engagement with the patient throughout the medical procedure;
a processor assembly including a patient monitoring signal receiver, said plurality of patient sensors transmitting a plurality of patient monitoring signals indicative of the plurality of physiologic parameters to said patient monitoring signal receiver throughout the medical procedure;
said processor assembly further comprising a patient signal processor to analyze said plurality of patient monitoring signals and to generate at least one environmental control signal and at least one sensory feedback signal based on said plurality of patient monitoring signals;
an environmental control assembly comprising at least one environmental control device disposed in communication with the controlled environment;
said processor assembly transmitting said at least one environmental control signal to said at least one environmental control device to control operation thereof;
a sensory feedback assembly comprising a sensory feedback device disposed in communication with the medical provider comprising a sensory feedback generator to provide a sensory feedback sensation to the medical provider based on said sensory feedback signal to alert the medical provider of the at least one physiologic parameter of the patient throughout the medical procedure; and
said processor assembly transmitting said sensory feedback signal to said sensory feedback device to control operation thereof.
